(19) United States
(12) Patent Application Publication
(10) Pub. No.: US 2010/0111947 A1
(43) Pub. Date: May 6, 2010

(54) PHARMACEUTICAL COMPOSITION COMPRISING ANTIBODY COMPOSITION WHICH SPECIFICALLY BINDS TO GANGLIOSIDE GM2

(75) Inventors: Toshihiko ISHII, Shizuoka (JP); Masao ASADA, Shizuoka (JP); Yukimasa SHIOTSU, Shizuoka (JP)

Correspondence Address: SUGHRUE-265550, 2100 PENNSYLVANIA AVE. NW, WASHINGTON, DC, 20037-3213, US

(73) Assignee: KYOWA HAKKO KIRIN CO., LTD., Tokyo (JP)

(21) Appl. No.: 12/540,533

(22) Filed: Aug. 13, 2009

Related U.S. Application Data
(60) Provisional application No. 61/089,222, filed on Aug. 15, 2008.

(30) Foreign Application Priority Data
Aug. 13, 2008 (JP) 2008-208383

(51) Int. Cl. A61K 39/395; A61P 35/00

(52) U.S. Cl. 424/133.1

(57) ABSTRACT

The present invention provides a pharmaceutical composition which is effective for treating multiple myeloma. The present invention relates to a pharmaceutical composition comprising a combination of an antibody composition which specifically binds to ganglioside GM2 or an antibody fragment thereof and at least one agent.

### BACKGROUND OF THE INVENTION

1. Field of the Invention

The present invention relates to a pharmaceutical composition comprising a combination of an antibody composition which specifically binds to ganglioside GM2 and at least one agent.

2. Background of the Invention

Ganglioside GM2 (hereinafter also referred to as GM2) is expressed on various tumor cells, such as kidney cancer, small cell lung cancer, glioma and multiple myeloma (hereinafter referred to as MM) (Non-patent Literatures 1 to 4).

Examples of a standard therapeutic agent against MM include Melphalan, Prednisolone and the like (Non-patent Literature 5). Furthermore, since Bortezomib, Thalidomide and Lenalidomide are recently approved, they are expected to be a standard agent for the treatment of MM in the future. However, although such drugs are newly introduced, a treatment with these drugs is not sufficient and it is still difficult to fully recover MM.

Excellent therapeutic results are sometimes obtained by combined use of a therapeutic antibody and an agent having low-molecular weight. For example, it has been reported that the combined use of anti-CD20 antibody, Rituximab, and agents for multidrug chemotherapy, such as CHOP (Non-patent Literature 6), the combined use of anti-VEGF antibody, Bevacizumab and multidrug chemotherapy, such as FOLFOX (Non-patent Literature 7) and the combined use of anti-ErbB2 antibody, Trastuzumab and taxane chemotherapy (Non-patent Literature 8) have significant effect on lymphoma, colon cancer and breast cancer, respectively.

Though various therapeutic antibodies are under development, no therapeutic agent for MM has been approved yet. In addition, as combined use of a therapeutic antibody and an agent having a low-molecular weight, a clinical trial was conducted to assess the combined use of Rituximab and Melphalan-prednisone (MP) therapy but it has been reported that it showed no advantage compared to a single drug (Non-patent Literature 9). At present, a method of combined use of anti-GM2 antibody and other agent which is clinically effective for treating MM is not known.

As an antibody which binds to ganglioside GM2, an anti-GM2 antibody which is a CDR-grafted antibody (Patent Literature 1), au anti-GM2 antibody having high antibody dependent cell-mediated cytotoxicity (hereinafter referred to as ADCC activity) (Patent Literature 2), au anti-GM2 antibody having high complement-dependent cytotoxicity (hereinafter referred to as CDC activity) (Patent Literature 3), an antibody against monosialo GM2 (Patent Literature 4, Patent Literature 5) and the like are known.

Patent Literature 1: U.S. Pat. No. 6,872,392

### Patent Literature 2: WO2005/035578

### Patent Literature 3: WO2008/090960

### Patent Literature 4: WO03/49704

### Patent Literature 5: WO04/53102

### Non-patent Literature 1: Kaushik, et al.; Cancer Res. 66, 6816-25, 2006

### Non-patent Literature 2: Nakamura, et al.; Cancer Res, 54, 1511-6, 1994

### Non-patent Literature 3: Dohi, et al.; Anticancer Res. 14, 2577-81, 1994

### Non-patent Literature 4: O'Boyle, et al.; Leukemia & Lymphoma, 21, 255-266, 1996

### Non-patent Literature 5: Barlogie, et al.; Blood, 103(1):20-32, 2004

### Non-patent Literature 6: Czuczman, et al.; J. Clin Oncol., 17, 268, 1999

### Non-patent Literature 7: Giantonio, et al.; J Clin Oncol., 25(12), 1539-44, 2007

### Non-patent Literature 8: Slamon, et al.; NEJM, Vol. 344, 783-792, 2001

### Non-patent Literature 9: Rachid, et al.; Leukemia and Lymphoma, 48, 2338-44, 2007

### SUMMARY OF THE INVENTION

An object of the present invention is to provide a pharmaceutical composition which is effective for treating multiple myeloma. The present invention relates to a pharmaceutical composition comprising a combination of an antibody composition which specifically binds to ganglioside GM2 or the antibody fragment thereof and at least one agent

### BRIEF DESCRIPTION OF THE DRAWINGS

FIG. 1 shows an effect provided by combined use of an anti-GM antibody and Melphalan against KMS-11 cells transplanted into a SCID mouse. The ordinate shows a V/V0 value. , □, Δ and ∘ indicate the average V/V0 values of a negative control group, a group administering KM8969 solely, a group administering Melphalan solely and a group administering KM8969 and Melphalan in combination.

FIG. 2 shows an effect provided by combined use of an anti-GM antibody and Bortezomib or Lenalidomide against OPM-2/GFP cells transplanted into in a SCID mouse. The ordinate shows a concentration of M protein in serum.

### DETAILED DESCRIPTION OF THE INVENTION

Specifically, the present invention relates to the following (1) to (11):

(1) A pharmaceutical composition comprising a combination of a recombinant antibody which specifically binds to ganglioside GM2 or an antibody fragment thereof and at least one agent;
(2) A pharmaceutical composition for administering a recombinant antibody which specifically binds to GM2 or an antibody fragment thereof and at least one agent in combination;
(3) A pharmaceutical composition for administering a recombinant antibody which specifically binds to GM2 or the antibody fragment thereof and at least one agent either simultaneously or successively;
(4) The pharmaceutical composition according to any one of the above (1) to (3), which is an antitumor agent;
(5) The pharmaceutical composition according to any one of the above (1) to (4), wherein the recombinant antibody which specifically binds to GM2 or the antibody fragment thereof has high ADCC activity;
(6) The pharmaceutical composition according to any one of the above (1) to (5), wherein the recombinant antibody which specifically binds to GM2 or the antibody fragment thereof has high CDC activity;
(7) The pharmaceutical composition according to any one of the above (1) to (6), wherein the agent is an agent having a low-molecular weight or an agent having a high-molecular weight;
(8) The pharmaceutical composition according to any one of the above (7), wherein the agent having a low-molecular weight is selected from the group consisting of a DNA synthesis inhibitor, a metabolic antagonist, an immuno-modulating agent, a proteasome inhibitor, a steroid agent, an HDAC inhibitor and an Hsp90 inhibitor;
(9) The pharmaceutical composition according to the above (4), the antitumor agent is a therapeutic agent for multiple myeloma;
(10) Use of a combination of a recombinant antibody which specifically binds to GM2 or an antibody fragment thereof and at least one agent in combination; and
(11) Use of a recombinant antibody which specifically binds to ganglioside GM2 or an antibody fragment thereof and at least one agent for manufacturer of a therapeutic agent for multiple myeloma.

Examples of the pharmaceutical composition in the present invention include a pharmaceutical composition comprising a combination of the recombinant antibody which specifically reacts with GM2 or the antibody fragment thereof and at least one agent, a pharmaceutical composition for administering the recombinant antibody which specifically reacts with GM2 or the antibody fragment thereof and at least one agent in combination, and a pharmaceutical composition for administering the recombinant antibody which specifically reacts with GM2 or the antibody fragment thereof and at least one agent either simultaneously or successively.

The pharmaceutical composition comprising a combination refers to a pharmaceutical composition in which the recombinant antibody which specifically binds to GM2 or the antibody fragment thereof and at least one agent are prepared separately and these ingredients are administered in combination either simultaneously or successively or a combined agent obtained by mixing each ingredient. The combined agent obtained by mixing each ingredient includes a fusion antibody obtained by binding at least one agent to the recombinant antibody which specifically binds to GM2, or the antibody fragment thereof, and the like.

In addition, a pharmaceutical kit including each ingredient is prepared and these ingredients are administered simultaneously or successively to a patient or administered to a patient after mixing them.

The recombinant antibody which specifically binds to GM2 and the antibody fragment thereof in the present invention (both of which are sometimes referred to as an antibody of the present invention) include a recombinant antibody which specifically reacts with GM2 and an antibody fragment thereof. Among these, a recombinant antibody having high ADCC activity or an antibody fragment thereof, and/or a recombinant antibody having high CDC activity or an antibody fragment thereof, and the like are preferable.

The recombinant antibody of the present invention includes a human chimeric antibody, a humanized antibody, a human antibody, and the like.

The human chimeric antibody refers to an antibody comprising H chain V region (hereinafter also referred to as HV or VH) and L chain V region (herein after also referred to as LV or VL) of an antibody of a non-human animal and CH and L chain C region (hereinafter also referred to as CL) of a human antibody. As the non-human animal, any animal, such as mouse, rat, hamster and rabbit, can be used so long as hybridomas can be prepared from the animal.

The human chimeric antibody of the present invention can be produced by obtaining cDNAs encoding VH and VL from a hybridoma capable of producing a monoclonal antibody derived from a non-human animal which specifically reacts with GM2, inserting them into an expression vector for animal cell having genes encoding human antibody CH and human antibody CL to thereby construct a vector for expression of a human chimeric antibody, and then introducing the vector into a host cell to express the antibody.

Any CH of a human chimeric antibody can be used, so long as it belongs to human immunoglobulin (hereinafter referred to as hIg), but those of IgG class are preferred, and any one of subclasses further belonging to IgG, such as γ1, γ2, γ3 and γ4, can be used. Also, as CL of a human chimeric antibody, those of κ class or λ class can be used.

The human chimeric antibody which specifically binds to GM2 of the present invention is anti-ganglioside GM2 chimeric antibody composition which comprises CDR1, CDR2 and CDR3 of VH comprising the amino acid sequences represented by SEQ ID NOs:1, 2 and 3, respectively, and/or CDR1, CDR2 and CDR3 of VL comprising the amino acid sequences represented by SEQ ID NOs:4, 5 and 6, respectively; anti-ganglioside GM2 chimeric antibody composition which comprises VH and VL comprising the amino acid sequences represented by SEQ ID NO:7 and 8, respectively; anti-ganglioside GM2 chimeric antibody composition wherein VH of the antibody comprises the amino acid sequence represented by SEQ ID NO:7, CH of the human antibody comprises an amino acid sequence of the hIgG1 subclass, VL of the antibody comprises the amino acid sequence represented by SEQ ID NO:8, and CL of the human antibody comprises an amino acid sequence of the κ class.

Specifically, the amino acid sequence of the human chimeric antibody composition of the present invention which specifically binds to ganglioside GM2 includes the amino acid sequence of KM966 described in EP598998.

More specifically, examples thereof include chimeric antibody DMF 10.167.4 and chimeric antibody ChGM2 described in WO03/49704 and WO04/53102 and the like.

A humanized antibody is an antibody in which amino acid sequences of CDRs of VH and VL of a non-human animal antibody are grafted into appropriate positions of VH and VL of a human antibody.

The humanized antibody of the present invention can be produced by constructing cDNAs encoding V regions in which the amino acid sequences of CDRs of VH and VL of a non-human animal antibody are grafted into the frameworks (hereinafter referred to as FR) of VH and VL of any human antibody, inserting them into an expression vector for animal cell comprising DNAs encoding CH and CL of a human antibody to thereby construct a humanized antibody expression vector, and then introducing the expression vector into an animal cell to express the humanized antibody.

The antibody derived from a non-human animal or the human chimeric antibody used for preparing the humanized antibody composition of the present invention includes mouse monoclonal antibody KM750 and mouse monoclonal antibody KM796 described in Japanese Published Unexamined Patent Application No. 311385/92, monoclonal antibody MoAb5-3 described in Cancer Res., 46, 4116 (1986), monoclonal antibody MK1-16 and monoclonal antibody MK2-34 described in Cancer Res., 48, 6154 (1988), monoclonal antibody DMAb-1 described in J. Biol. Chem., 264, 12122 (1989), chimeric antibody DMF 10.167.4 and chimeric antibody ChGM2 described in WO2003/049704 and WO04/53102 and the like.

As the FR amino acid sequences of VH and VL of a humanized antibody, any of those derived from human antibodies can be used. Suitable sequences include the FR amino acid sequences of VH and VL of human antibodies registered in databases such as Protein Data Bank, and the amino acid sequences common to all FR subgroups of VH and VL of human antibodies [Sequences of Proteins Immunological Interest, US Dept. Health and Human Services (1991)].

As the CH of the humanized antibody, any CH can be used, so Long as it belongs to the hIg, and those of the hIgG class are preferred and any one of the subclasses belonging to the hIgG class, such as γ1, γ2, γ3 and γ4, can be used. In addition, as the CL of the humanized antibody, any CL can be used, so long as it belongs to the big class, and those belonging to the κ class or λ class can be used.

Example of the humanized antibody composition of the present invention include a humanized antibody composition comprising CDRs of VH and VL of an antibody derived from a non-human animal which specifically reacts with ganglioside GM2 and the like. Preferable examples include a humanized antibody composition or antibody fragment composition comprising CDR1, CDR2 and CDR3 of VH comprising the amino acid sequences represented by SEQ ID NOs:1, 2 and 3, respectively, and/or CDR1, CDR2 and CDR3 of VL comprising the amino acid sequences represented by SEQ ID NOs:4, 5 and 6, respectively, and the like.

Among these humanized antibody compositions, preferred humanized antibody compositions include: a humanized antibody composition, wherein VH of the antibody comprises an amino acid sequence in which at least one amino acid residue selected from the group consisting of Arg at position 38, Ala at position 40, Gln at position 43 and Gly at position 44 is substituted with other amino acid residue in the amino acid sequence represented by SEQ ID NO:9; a humanized antibody composition, wherein VH of the antibody comprises an amino acid sequence in which at least one amino acid residue selected from the group consisting of Arg at position 67, Ala at position 72, Ser at position 84 and Arg at position 98 is substituted with other amino acid residue in the amino acid sequence represented by SEQ ID NO:10; a humanized antibody composition, wherein VL of the antibody comprises an amino acid sequence in which at least one amino acid residue selected from the group consisting of Val at position 15, Tyr at position 35, Leu at position 46, Ser at position 59, Asp at position 69, Phe at position 70, Thr at position 71, Phe at position 72 and Ser at position 76 is substituted with other amino acid residue in the amino acid sequence represented by SEQ ID NO:11; and a humanized antibody composition, wherein VL of the antibody comprises an amino acid sequence in which at least one amino acid residue selected from the group consisting of Met at position 4, Leu at position 11, Val at position 15, Tyr at position 35, Ala at position 42, Len at position 46, Asp at position 69, Phe at position 70, Thr at position 71, Leu at position 77 and Vat at position 103 is substituted with other amino acid residue in the amino acid sequence represented by SEQ ID NO:12. More preferred are the following antibody compositions: a humanized antibody composition, wherein VH of the antibody comprises an amino acid sequence in which at least one amino acid residue selected from the group consisting of Arg at position 38, Ala at position 40, Gln at position 43 and Gly at position 44 is substituted with other amino acid residue in the amino acid sequence represented by SEQ ID NO:9, and VL of the antibody comprises an amino acid sequence in which at least one amino acid residue selected from the group consisting of Val at position 15, Tyr at position 35, Leu at position 46, Ser at position 59, Asp at position 69, Phe at position 70, Thr at position 71, Phe at position 72 and Ser at position 76 is substituted with other amino acid residue in the amino acid sequence represented by SEQ ID NO:11; a humanized antibody composition, wherein VH of the antibody comprises an amino acid sequence in which at least one amino acid residue selected from the group consisting of Arg at position 67, Ala at position 72, Ser at position 84 and Arg at position 98 is substituted with other amino acid residue in the amino acid sequence represented by SEQ ID NO:10, and VL of the antibody comprises an amino acid sequence in which at least one amino acid residue selected from the group consisting of Val at position 15, Tyr at position 35, Leu at position 46, Ser at position 59, Asp at position 69, Phe at position 70, Thr at position 71, Phe at position 72 and Ser at position 76 is substituted with other amino acid residue in the amino acid sequence represented by SEQ ID NO:11; and a humanized antibody composition, wherein VH of the antibody comprises an amino acid sequence in which at least one amino acid residue selected from the group consisting of Arg at position 67, Ala at position 72, Ser at position 84 and Arg at position 98 is substituted with other amino acid residue in the amino acid sequence represented by SEQ ID NO:10, and VL of the antibody comprises an amino acid sequence in which at least one amino acid residue selected from the group consisting of Met at position 4, Leu at position 11, Val at position 15, Tyr at position 35, Ala at position 42, Leu at position 46, Asp at position 69, Phe at position TO, Thr at position 71, Leu at position 77 and Val at position 103 is substituted with other amino acid residue in the amino acid sequence represented by SEQ ID NO:12.

Specific examples of the humanized antibody composition include a humanized antibody composition wherein VH of the antibody comprises an amino acid sequence selected from the group consisting of the amino acid sequences represented by SEQ ID NOs:9, 10, 13, 14, 15, 16 and 17, an amino acid sequence in which at least one amino acid residue selected from the group consisting of Arg at position 38, Ala at position 40, Gln at position 43 and Gly at position 44 is substituted with other amino acid residue in the amino acid sequence represented by SEQ ID NO:9, and an amino acid sequence in which at least one amino acid residue selected from the group consisting of Arg at position 67, Ala at position 72, Ser at position 84 and Arg at position 98 is substituted with other amino acid residue in the amino acid sequence represented by SEQ ID NO:10; and/or VL of the antibody comprises an amino acid sequence selected from the group consisting of the amino acid sequences represented by SEQ ID NOs:11, 12, 18, 19, 20, 21 and 22, an amino acid sequence in which at least one amino acid residue selected from the group consisting of Val at position 15, Tyr at position 35, Leu at position 46, See at position 59, Asp at position 69, Phe at position 70, Thr at position 71, Phe at position 72 and Ser at position 76 is substituted with other amino acid residue in the amino acid sequence represented by SEQ ID NO:11, and an amino acid sequence in which at least one amino acid residue selected from the group consisting of Met at position 4, Leu at position 11, Val at position 15, Tyr at position 35, Ala at position 42, Leu at position 46, Asp at position 69, Phe at position 70, Thr at position 71, Leu at position 77 and Val at position 103 is substituted with other amino acid residue in the amino acid sequence represented by SEQ ID NO:12. Specific examples include a humanized antibody composition wherein VH of the antibody comprises an amino acid sequence represented by SEQ ID NO:13 and VL of the antibody comprises an amino acid sequence represented by SEQ ID NOs:18 or 19; and a humanized antibody composition wherein VH of the antibody comprises the amino acid sequences represented by SEQ ID NO:9, and VL of the antibody comprises the amino acid sequence represented by SEQ ID NO:19 or 22.

The humanized antibody composition of the present invention is most preferably a humanized antibody composition wherein VH of the antibody comprises the amino acid sequence represented by SEQ ID NO:13, and VL of the antibody comprises the amino acid sequence represented by SEQ ID NO:18; or a humanized antibody composition wherein VH of the antibody comprises the amino acid sequence represented by SEQ ID NO:9, and VL of the antibody comprises the amino acid sequence represented by SEQ ID NO:19.

Examples of the amino acid sequence of the antibody variable region of the humanized antibody composition of the present invention include amino acid sequences of antibody variable regions of KM8966 produced by transformant KM8966 (FERM BP-5105), KM8967 produced by transformant KM8967 (FERM BP-5106), KM8969 produced by transformant KM8969 (FERM BP-5527) and KM8970 produced by transformant KM8970 (FERM BP-5528) each described in Japanese Published Unexamined Patent Application No. 257893/98 and the like.

The antibody composition of the present invention also includes antibodies and antibody fragments which specifically bind to ganglioside GM2, and consist of amino acid sequences wherein one or more amino acid residue(s) is/are deleted, added, substituted and/or inserted in the above amino acid sequences.

In the amino acid sequences of the antibody composition of the present invention, the number of amino acid residues which are deleted, substituted, inserted and/or added is one or more and is not specifically limited, but it is within the range where deletion, substitution or addition is possible by known methods such as site-directed mutagenesis described in Molecular Cloning, Second Edition; Current Protocols in Molecular Biology; Nucleic Acids Research, 10, 6487 (1982); Proc. Natl. Acad. Sci. USA, 79, 6409 (1982); Gene, 34, 315 (1985); Nucleic Acids Research, 13, 4431 (1985); Proc. Natl. Acad. Sci. USA, 82, 488 (1985), etc. The suitable number is 1 to dozens, preferably 1 to 20, more preferably 1 to 10, further preferably 1 to 5.

The expression “one or more amino acid residue(s) is/are deleted, substituted, inserted or added in the amino acid sequence of the antibody composition of the present invention” means that the amino acid sequence of the antibody composition contains deletion, substitution, insertion or addition of a single or plural amino acid residues in a single or plural amino acid sequences at arbitrary positions therein. Deletion, substitution, insertion or addition may be simultaneously contained in one sequence, and amino acid residues to be substituted, inserted or added may be either natural or not. Examples of the natural amino acid residues are L-alanine, L-asparagine, L-aspartic acid, L-glutamine, L-glutamic acid, glycine, L-histidine, L-isoleucine, L-leucine, L-lysine, L-methionine, L-phenylalanine, L-proline, L-serine, L-threonine, L-tryptophan, L-tyrosine, L-valine, L-cysteine and the like.

The followings are preferred examples of the amino acid residues capable of mutual substitution. The amino acid residues in the same group shown below can be mutually substituted.

Group A: leucine, isoleucine, norleucine, valine, norvaline, alanine, 2-aminobutanoic acid, methionine, O-methylserine, t-butylglycine, t-butylalanine, cyclohexylalanine
Group B: aspartic acid, glutamic acid, isoaspartic acid, isoglutamic acid, 2 aminoadipic acid, 2-aminosuberic acid
Group C: asparagine, glutamine
Group D: lysine, arginine, ornithine, 2,4-diaminobutanoic acid, 2,3-diaminopropionic acid
Group E: proline, 3-hydroxyproline, 4-hydroxyproline
Group F: serine, threonine, homoserine
Group G: phenylalanine, tyrosine

A human antibody is originally an antibody naturally existing in the human body, and it also includes antibodies obtained from a human antibody phage library or a human antibody-producing transgenic animal, which is prepared based on the recent advance in genetic engineering, cell engineering and developmental engineering techniques.

The antibody existing in the human body can be prepared, for example by isolating a human peripheral blood lymphocyte, immortalizing it by infecting with EB virus or the like and then cloning it to thereby obtain lymphocytes capable of producing the antibody, culturing the lymphocytes thus obtained, and purifying the antibody from the supernatant of the culture.

The human antibody phage library is a library in which antibody fragments, such as Fab and scFv, are expressed on the phage surface by inserting a gene encoding an antibody prepared from a human B cell into a phage gene. A phage expressing an antibody fragment having the desired antigen binding activity can be recovered from the library, using its activity to bind to an antigen-immobilized substrate as the index. The antibody fragment can be converted further into a human antibody molecule comprising two full H chains and two full L chains by genetic engineering techniques.

A human antibody-producing transgenic animal is an animal in which a human antibody gene is integrated into cells. Specifically, a human antibody-producing transgenic animal can be prepared by introducing a gene encoding a human antibody into a mouse ES cell, grafting the ES cell into an early stage embryo of other mouse and then developing it. A human antibody is prepared from the human antibody-producing transgenic non-human animal by obtaining a human antibody-producing hybridoma by a hybridoma preparation method usually carried out in non-human mammals, culturing the obtained hybridoma and forming and accumulating the human antibody in the supernatant of the culture.

Examples of the antibody fragment composition having binding activity to a target molecule include Fab, Fab, F(ab′)₂, scFv, diabody, dsFv and a peptide comprising CDR.

An Fab is an antibody fragment having a molecular weight of about 50,000 and having antigen binding activity, in which about a half of the N-terminal side of H chain and the entire L chain, among fragments obtained by treating IgG with a protease, papain (cleaving an amino acid residue at the 224th position of the H chain), are bound together through a disulfide bond (S—S bond).

An F(ab′)₂ is an antibody fragment having antigen binding activity and having a molecular weight of about 100,000 which is somewhat larger than one in which Fab are bound via an S—S bond in the hinge region, among fragments obtained by treating IgG with a protease, pepsin (by cleaving the H chain at the 234th amino acid residue).

An Fab′ is an antibody fragment having a molecular weight of about 50,000 and having antigen binding activity, which is obtained by cleaving an S—S bond in the hinge region of the F(ab′)₂.

An scFv is a VH-P-VL or VL-P-VH polypeptide in which one chain VH and one chain VL are linked using an appropriate peptide linker (P) having 12 or more residues and is an antibody fragment having antigen binding activity.

A diabody is an antibody fragment in which scFv's having the same or different antigen binding specificity forms a dimer, and has divalent antigen binding activity to the same antigen or two specific antigen binding activities to different antigens.

A dsFv is obtained by binding polypeptides in which one amino acid residue of each of VH and VL is substituted with a cysteine residue via an S—S bond between the cysteine residues.

A peptide comprising CDR is constituted by including at least one region or more of CDRs of VH or VL. Plural peptide comprising CDRs can be produced by binding directly or via an appropriate peptide linker.

In addition, examples of the recombinant antibody which specifically binds to ganglioside GM2 used in the present invention include a recombinant antibody which specifically binds to ganglioside GM2 and has high ADCC activity and/or a recombinant antibody which specifically binds to ganglioside GM2 and has high CDC activity and the like.

In the present invention, examples of the recombinant antibody which specifically binds to ganglioside GM2 and has high ADCC activity include a recombinant antibody composition which comprises antibody molecules having N-glycoside-linked sugar chains in the Fc region, and binds to ganglioside GM2 having sugar chains in which fucose is not bound to N-acetylglucosamine in the reducing end in the sugar chains, among the total complex type N-glycoside-linked sugar chains which bind to the Fe region in the composition.

An antibody molecule has the Fc region, to which N-glycoside-linked sugar chains are bound. Therefore, two sugar chains are bound to one antibody molecule.

The N-glycoside-linked sugar chains include complex type sugar chains having one or multiple number of parallel galactose-N-acetylglucosamine (hereinafter referred to as Gal-GlcNAc) side chains in the non-reducing end of the core structure and having sialic acid, bisecting N-acetylglucosamine or the like in the non-reducing end of Gal-GlcNAc.

In the present invention, the complex type N-glycoside-linked sugar chain is represented by the following chemical formula.

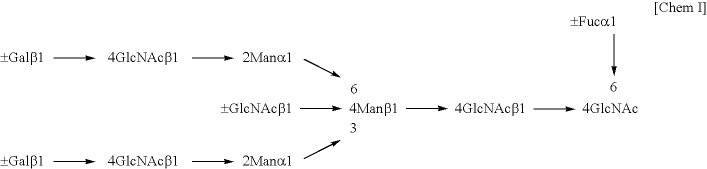

Among the recombinant antibody compositions which specifically bind to ganglioside GM2 of the present invention, the recombinant antibody composition comprising antibody molecules having the N-glycoside-linked sugar chains in the Fe region may comprise an antibody molecule having the same sugar chain structure or antibody molecules having different sugar chain structures, so long as it has the above sugar chain structure. That is, the recombinant antibody composition of the present invention means a composition comprising recombinant antibody molecules having the same or different sugar chain structure(s).

A ratio of sugar chains to which fucose is not bound to N-acetylglucosamine in the reducing end is not limited, so long as ADCC activity is enhanced. The ratio is preferably 20% or more, more preferably 51% -100%, further preferably 80% -100%, especially preferably 90% -99%, and most preferably 100%.

In the present invention, the sugar chain in which fucose is not bound may have any sugar chain structure in the non-reducing end, so long as fucose is not bound to N-acetylglucosamine in the reducing terminal in the above formula.

In the present invention, the case where fucose is not bound to N-acetylglucosamine in the reducing end in the sugar chains means that fucose is not substantially bound. An antibody composition in which fucose is not substantially bound specifically refers to an antibody composition in which fucose is not substantially detected, when subjected to the conventional sugar chain analysis (WO02/31140 and WO03/85107). An extent that fucose is not substantially detected specifically refers to an extent that fucose is below the detection limit of measurement A recombinant antibody composition in which fucose is not bound to N-acetylglucosamine in the reducing ends of all sugar chains has highest ADCC activity.

The ratio of antibody molecules having sugar chains in which fucose is not bound to N-acetylglucosamine in the reducing end in the sugar chains contained in the composition which comprises antibody molecules having complex type N-glycoside-linked sugar chains in the Fc region can be determined by the following analysis methods.

Examples of the analysis method include the method comprising releasing the sugar chains from the antibody molecule using a known method such as hydrazinolysis or enzyme digestion [Biochemical Experimentation Methods 23—Method for Studying Glycoprotein Sugar Chain (Japan Scientific Societies Press), edited by Reiko Takahashi (1989)], carrying out fluorescence labeling or radioisotope labeling of the released sugar chains and then separating the labeled sugar chains by chromatography; the method comprising analyzing the released sugar chains with the HPAED-PAD method [J. Liq. Chromatogr., 6, 1577 (1983)], and the Like.

The transformant producing the recombinant antibody composition which specifically binds to ganglioside GM2 of the present invention can be obtained by introducing, into an animal cell, a recombinant antibody composition expression vector into which DNAs encoding a variable region and a constant region of an antibody molecule are inserted.

The recombinant antibody composition expression vector is constructed as below (WO02/31140, WO03/85107).

Each of the above DNAs encoding CH and CL is introduced into an expression vector for animal cell to produce an expression vector for animal cell.

The expression vector for animal cell includes pAGE107 (Japanese Published Unexamined Patent Application No. 22979/91; Miyaji H. et al., Cytotechnology, 3, 133-140 (1990)), pAGE103 (Mizukami T. and Itoh S., J. Biochem., 101, 1307-1310 (1987)), pHSG274 (Brady G et al., Gene, 27, 223-232 (1984)), pKCR (O'Hare K. et al., Proc. Natl. Acad. Sci. USA., 78, 1527-1531 (1981)), pSG1βd2-4 (Miyaji H. at al., Cytotechnology, 4, 173-180 (1990)) and the Like. The promoter and enhancer used for the expression vector for animal cell include SV40 early promoter and enhancer (Mizukami T. and Itoh S., J. Biochem., 101, 1307-1310 (1987)), LTR promoter and enhancer of Moloney mouse leukemia virus (Kuwana Y. et al., Biochem. Biophys. Res. Commun., 149, 960-968 (1987)), immunoglobulin H chain promoter (Mason J. O. et al., Cell, 41, 479-487 (1985)) and enhancer (Gillies S. D. et al., Cell, 33, 717-728 (1983)) and the like.

The vector for expression of recombinant antibody composition may be either of a type in which genes encoding the H chain and L chain exist on separate vectors or of a type in which both genes exist on the same vector (tandem type). In respect of easiness of construction of a recombinant antibody composition expression vector, easiness of introduction into animal cells, and balance between the expression amounts of the H and L chains of an antibody in animal cells, a tandem type of the vector for expression of recombinant antibody composition is preferred (Shitara K. et al., Immunol. Methods, 167, 271-278 (1994)). The tandem type vector for expression of recombinant antibody composition includes pKANTEX93 (WO97/10354), pEE18 (Bentley K. J. et al., Hybridoma, 17, 559-567 (1998)) and the like.

cDNAs encoding VH and VL of antibodies for various antigens are cloned into the upstream of DNAs encoding CH and CL of the constructed vector for expression of recombinant antibody composition to thereby construct a recombinant antibody composition expression vector.

A method for introducing the expression vector into a host cell includes electroporation (Japanese Published Unexamined Patent Application No. 257391-90; Miyaji H. et al., Cytotechnoloy, 3, 133-140 (1990)) and the like.

The host cell producing the recombinant antibody composition of the present invention may be any host cell, such as an animal cell, a plant cell or a microorganism, so long as it is generally used in production of a recombinant protein.

The host cell producing the recombinant antibody composition of the present invention includes a CHO cell derived from a Chinese hamster ovary tissue, a rat myeloma cell line YB2/3HL.P2.G11.16Ag.20 cell, a mouse myeloma cell line NS0 cell, a mouse myeloma SP2/0-Ag14 cell, a BHK cell derived from a Syrian hamster kidney tissue, a human leukemia cell Line Namalwa cell, a hybridoma cell produced by using a myeloma cell and any B cell, a hybridoma cell produced by a B cell obtained by immunizing with an antigen a transgenic non-human animal produced by using an embryonic stem cell or a fertilized egg cell and any myeloma cell; a hybridoma cell produced by the above myeloma cell and a B cell obtained by immunizing with an antigen a transgenic non-human animal produced by using an embryonic stem cell or a fertilized egg cell and the like.

The host cell capable of expressing a recombinant antibody composition having high ADCC activity includes a host cell resistant to a lectin which recognizes a sugar chain structure in which 1-position of fucose is bound to 6-position of N-acetylglucosamine in the reducing end through α-bond in the complex type N-glycoside-linked sugar chain, such as a host cell capable of producing an antibody composition comprising an antibody molecule having complex type N-glycoside-linked sugar chains in the Fc region, wherein the ratio of sugar chains in which fucose is not bound to N-acetylglucosamine in the reducing end of the sugar chains among the total complex type N-glycoside-linked sugar chains which bind to the Fe region contained in the composition is 20% or more. Examples include cells in which activity of at least one protein described below is decreased or deleted, and the like:

(a) an enzyme relating to synthesis of an intracellular sugar nucleotide, GDP-fucose;
(b) an enzyme relating to the modification of a sugar chain in which 1-position of fucose is bound to 6-position of N-acetylglucosamine in the reducing end through α-bond in a complex type N-glycoside-linked sugar chain;
(c) a protein relating to transport of an intracellular sugar nucleotide, GDP-fucose, to the Golgi body (WO02/31140, WO03/85107).

The above host cell is preferably a host cell in which a gene encoding α1,6-fucosyltransferase in the host cell is knocked out (WO02/31140, WO03/85107).

The enzyme relating to synthesis of an intracellular sugar nucleotide, GDP-fucose may be any enzyme, so long as it is an enzyme relating to the synthesis of the intracellular sugar nucleotide, GDP-fucose, as a supply source of fucose to a sugar chain. The enzyme relating to synthesis of an intracellular sugar nucleotide, GDP-fucose includes an enzyme which has influence on the synthesis of the intracellular sugar nucleotide, GDP-fucose, and the like.

The intracellular sugar nucleotide, GDP-fucose, is supplied by a de novo synthesis pathway or Salvage synthesis pathway. Thus, all enzymes relating to the synthesis pathways are included in the enzyme relating to synthesis of an intracellular sugar nucleotide, GDP-fucose.

The enzyme relating to the de novo synthesis pathway of an intracellular sugar nucleotide, GDP-fucose includes GDP-mannose 4,6-dehydratase (hereinafter referred to as GMD), GDP-keto-6-deoxymannose-3,5-epimerase, 4,6-reductase (hereinafter referred to as Fx) and the like.

The enzyme relating to the salvage synthesis pathway of an intracellular sugar nucleotide, GDP-fucose includes GDP-beta-L-fucose pyrophosphorylase (hereinafter referred to as GFPP), Fucokinase and the like.

The enzyme which has influence on the synthesis of an intracellular sugar nucleotide, GDP-fucose also include an enzyme which has influence on the activity of the enzyme relating to the synthesis pathway of the intracellular sugar nucleotide, GDP-fucose described above, and an enzyme which has influence on the structure of substances as the substrate of the enzyme.

The enzyme relating to the modification of a sugar chain in which 1-position of fucose is bound to 6-position of N-acetylglucosamine in the reducing end through α-bond in a complex type N-glycoside-linked sugar chain includes any enzyme, so long as it is an enzyme relating to the reaction of binding of 1-position of fucose to 6-position of N-acetylglucosamine in the reducing end through α-bond in the complex type N-glycoside-linked sugar chain. The enzyme relating to the reaction of binding of 1-position of fucose to 6-position of N-acetylglucosamine in the reducing end through α-bond in the complex type N-glycoside-linked sugar chain includes an enzyme which has influence on the reaction of binding of 1-position of fucose to 6-position of N-acetylglucosamine in the reducing end through α-bond in the complex type N-glycoside-linked sugar chain. Examples include α-1,6-fucosyltransferase, α-L-fucosidase and the like.

Also, the enzyme relating to the reaction of binding of 1-position of fucose to 6-position of N-acetylglucosamine in the reducing end through α-bond in the complex type N-glycoside-linked sugar chain includes an enzyme which has influence on the activity of the enzyme relating to the reaction of binding of 1-position of fucose to 6-position of N-acetylglucosamine in the reducing end through α-bond in the complex type N-glycoside-linked sugar chain and an enzyme which has influence on the structure of substances as the substrate of the enzyme.

The protein relating to transport of an intracellular sugar nucleotide, GDP-fucose, to the Golgi body may be any protein, so long as it is a protein relating to the transport of the intracellular sugar nucleotide, GDP-fucose, to the Golgi body, or a protein which has an influence on the reaction for the transport of the intracellular sugar nucleotide, GDP-fucose, to the Golgi body.

The protein relating to the transport of the intracellular sugar nucleotide, GDP-fucose, to the Golgi body includes a GDP-fucose transporter and the like.

Also, examples of the protein which has an influence on the reaction for the transport of the intracellular sugar nucleotide, GDP-fucose, to the Golgi body include a protein which has an influence on the activity of the above protein relating to the transport of the intracellular sugar nucleotide, GDP-fucose, to the Golgi body or has influence on the expression thereof.

The method for obtaining a cell in which the above enzyme activity is decreased or deleted may be any method, so long as it is a method for decreasing or deleting the objective enzyme activity. Examples include:

(a) gene disruption targeting at a gene encoding the enzyme;
(b) introduction of a dominant-negative mutant of a gene encoding the enzyme;
(c) introduction of a mutation into the enzyme;
(d) suppression of transcription or translation of a gene encoding the enzyme;
(e) selection of a cell line resistant to a lectin which recognizes a sugar chain structure in which 1-position of fucose is bound to 6-position of N-acetylglucosamine in the reducing end through α-bond in a N-glycoside-linked sugar chain (WO02/31140, WO03/85107); and the like.

As the lectin which recognizes a sugar chain structure in which 1-position of fucose is bound to 6-position of N-acetylglucosamine in the reducing end through α-bond in a N-glycoside-linked sugar chain, any lectin capable of recognizing the sugar chain structure can be used. Specific examples include Lentil lectin LCA (lentil agglutinin derived from Lens culinaris), pea lectin PSA (pea lectin derived from Pisum sativum), broad bean lectin VFA (agglutinin derived from Vicia faba), Aleuria aurantia lectin AAL (lectin derived from Aleuria aurantia) and the like.

The “cell resistant to a lectin” refers to a cell in which growth is not inhibited by the presence of a lectin at an effective concentration. The “effective concentration” is a concentration higher than the concentration that does not allow the normal growth of a cell prior to the genome modification (hereinafter referred to also as parent cell line), preferably equal to the concentration that does not allow the normal growth of a cell prior to the genome modification, more preferably 2 to 5 times, further preferably 10 times, most preferably 20 or more times the concentration that does not allow the normal growth of a cell prior to the modification of the genomic gene.

The effective concentration of lectin that does not inhibit growth may be appropriately determined according to each cell line. It is usually 10 μml to 10 mg/ml, preferably 0.5 mg/ml to 2.0 mg/ml.

In the present invention, the recombinant antibody which specifically binds to ganglioside GM2 and has high CDC activity of the present invention may be any antibody composition, so long as it is a recombinant antibody composition having higher complement-dependent cytotoxic activity than a human IgG1 antibody and a human IgG3 antibody, wherein a polypeptide comprising a CH2 domain in the Fe region of a human IgG1 antibody is replaced by a polypeptide comprising an amino acid sequence which corresponds to the same position of a human IgG3 antibody indicated by the EU index as in Kabat et al., among the recombinant antibody compositions in which domains of CH1, the hinge, CH2 and CH3 in the heavy chain constant region of a human IgG1 are swapped into domains corresponding to IgG3 (hereinafter referred to as domain-swapped antibody).

An antibody molecule is constituted by polypeptides called heavy chain (hereinafter referred to as H chain) and light chain (hereinafter referred to as L chain). Also, the H chain is constituted by regions of a heavy chain variable region (VH) and a heavy chain constant region (hereinafter referred to as CH) from its N-terminal, and the L chain is constituted by regions of a light chain variable region (hereinafter referred to as VL) and a light chain constant region (hereinafter referred to as CL) from its N-terminal. CH is further constituted by domains of a CH1 domain, a hinge domain, a CH2 domain and a CH3 domain. The domain means a functional constitution unit constituting each polypeptide in the antibody molecule. Also, the CH2 domain and the CH3 domain in combination are called Fc region.

The CH1 domain, the hinge domain, the CH2 domain, the CH3 domain and the Fc region of the present invention are defined by positions of amino acid residues from the N-terminal indicated by the EU index as in Kabat, et al. [Sequence of Proteins of Immunological Interest, 5th Edition (1991)]. Specifically, CH1 is defined as the amino acid sequence of positions 118 to 215 indicated by the EU index, the hinge is defined as the amino acid sequence of positions 216 to 230 indicated by the EU index, CH2 is defined as the amino acid sequence of positions 231 to 340 indicated by the EU index, and CH3 is defined as the amino acid sequence of positions 341 to 447 indicated by the EU index.

Specifically, the recombinant antibody composition which specifically binds to ganglioside GM2 of the present invention includes a recombinant composition in which the polypeptide comprising a CH2 domain in the Fe region of a human IgG1 antibody is a polypeptide selected from the following (a) to (j);

(a) a polypeptide comprising the amino acid sequence at positions 231 to 340 of an IgG1 antibody indicated by the EU index;
(b) a polypeptide comprising the amino acid sequence at positions 231 to 356 of an IgG1 antibody indicated by the EU index;
(c) a polypeptide comprising the amino acid sequence at positions 231 to 358 of an IgG1 antibody indicated by the EU index;
(d) a polypeptide comprising the amino acid sequence at positions 231 to 384 of an IgG 1 antibody indicated by the EU index;
(e) a polypeptide comprising the amino acid sequence at positions 231 to 392 of an IgG1 antibody indicated by the EU index;
(f) a polypeptide comprising the amino acid sequence at positions 231 to 397 of an IgG1 antibody indicated by the EU index;
(g) a polypeptide comprising the amino acid sequence at positions 231 to 422 of an IgG1 antibody indicated by the EU index;
(h) a polypeptide comprising the amino acid sequences at positions 231 to 434 and at positions 436 to 447 of an IgG1 antibody indicated by the EU index;
(i) a polypeptide comprising the amino acid sequence at positions 231 to 435 of an IgG1 antibody indicated by the EU index; and
(j) a polypeptide comprising the amino acid sequence at positions 231 to 447 of an IgG1 antibody indicated by the EU index.

The amino acid sequence of the CL region in the recombinant antibody composition of the present invention may be either an amino acid sequence of a human antibody or an amino acid sequence from a non-human animal, but it is preferably Cκ or Cλ of an amino acid sequence of a human antibody.

Accordingly, the recombinant antibody composition which specifically binds to ganglioside GM2 of the present invention includes a recombinant antibody composition in which a polypeptide comprising a CH2 domain in the Fc region is replaced with a polypeptide comprising an amino acid sequence which corresponds to the same position of a human IgG3 antibody indicated by the EU index has higher CDC activity than a human IgG1 antibody and a human IgG3 antibody.

Furthermore, the recombinant antibody composition which specifically binds to ganglioside GM2 of the present invention includes a recombinant antibody composition having higher complement-dependent cytotoxic activity than a human IgG1 antibody and a human IgG3 antibody and having binding activity to protein A, which is substantially equal to that of a human IgG1 antibody, wherein a polypeptide comprising a CH2 domain in the Fc region of a human IgG1 antibody is replaced by a polypeptide comprising an amino acid sequence which corresponds to the same position of a human IgG3 antibody indicated by the EU index as in Kabat, et al.

Specifically, the recombinant antibody composition of the present invention include a recombinant composition in which the polypeptide comprising a CH2 domain in the Fe region of a human IgG1 antibody is a polypeptide selected from the following (a) to (h):

(a) a polypeptide comprising the amino acid sequence at positions 231 to 340 of an IgG1 antibody indicated by the EU index;
(b) a polypeptide comprising the amino acid sequence at positions 231 to 356 of an IgG1 antibody indicated by the EU index;
(c) a polypeptide comprising the amino acid sequence at positions 231 to 358 of an IgG1 antibody indicated by the EU index;
(d) a polypeptide comprising the amino acid sequence at positions 231 to 384 of an IgG1 antibody indicated by the EU index;
(e) a polypeptide comprising the amino acid sequence at positions 231 to 392 of an IgG1 antibody indicated by the EU index;
(f) a polypeptide comprising the amino acid sequence at positions 231 to 397 of an IgG1 antibody indicated by the EU index;
(g) a polypeptide comprising the amino acid sequence at positions 231 to 422 of an IgG1 antibody indicated by the EU index; and
(h) a polypeptide comprising the amino acid sequences at positions 231 to 434 and at positions 436 to 447 of an IgG1 antibody indicated by the EU index.

The binding activity to protein A can be measured by ELISA, surface plasmon resonance or the like. Specifically, the antibody composition is allowed to react with protein A solid-phased on a plate and then is further allowed to react with an antibody which recognizes the variously labeled antibodies, and the binding activity can be measured by determining the antibody composition bound to protein A.

Also, the antibody composition is allowed to react with protein A bound to a carrier such as sepharose at high pH conditions such as a pH of about 5 to 8, followed by washing, and then the binding activity can be measured by determining the antibody composition eluted at low pH conditions such as a pH of about 2 to 5.

Agents used in the present invention include an agent having a low-molecular weight, an agent having a high-molecular weight and the like.

Examples of the agent having a low-molecular weight include a DNA synthesis inhibitor, a mitotic inhibitor, a metabolic antagonist, an immuno-modulating agent, a proteasome inhibitor, a steroid agent, an HDAC inhibitor, an HSP90 inhibitor and the like.

Examples of the DNA synthesis inhibitor include Melphalan, Cyclophosphamide, Doxorubicin, Liposomal Doxorubicin, Etoposide, Cisplatin, Bendamustine and the like. Among these, Melphalan, is preferred.

Examples of the mitotic inhibitor include Vincristine.

Examples of the metabolic antagonist include Fludarabine.

Examples of the immuno-modulating agent include Thalidomide, Lenalidomide, and Pomalidomide. Among these, Lenalidomide is preferred.

Examples of the proteasome inhibitor include Bortezomib, Carfilzomib, and NPI-0052 (salinosporamide A).

Examples of the steroid include Prednisone, Dexamethasone and the like.

Examples of the HDAC inhibitor include vorinostat, panobinostat and the like.

Examples of the Hsp 90 inhibitor include tanespimycin and the like.

Examples of the agent having a low-molecular weight include Melphalan, Cyclophosphamide, Doxorubicin, Liposomal Doxorubicin, Etoposide, Cisplatin, Bendamustine, Vincristine, Fludarabine, Thalidomide, Lenalidomide, Pomalidomide, Bortezomib, Carfilzomib, NPI-0052 (salinosporamide A), Prednisone, Dexamethasone, vorinostat, panobinostat and tanespimycin or derivatives thereof, or a combination thereof.

As an agent having a low-molecular weight, Melphalan, Lenalidomide or Bortezomib, or a combination thereof is preferred.

Examples of the agents having a high-molecular weight include protein and the like. Examples of the protein include cytokines, antibodies and the like.

Examples of the cytokines include cytokines which activate the effector cells such as NK cells, macrophages, monocytes, and granulocytes, which are immunocompetent cells and derivatives thereof. Specific examples of the cytokines include interleukin 2 (IL-2), IFN-α, IFN-γ, IL-12, IL-15, IL-18, IL-21, fractalkine, M-CSF, GM-CSF, G-CSF, TNF-α, TNF-β, IL-1α, IL-1β, and the like.

Examples of the antibodies include a therapeutic antibody for treating a disease for which a recombinant antibody which specifically binds to GM2 used in the present invention, is targeted and an antibody which enhances immune activity; an antibody fragment thereof, and a fusion antibody thereof. In the case of the targeted disease for the antibody of the present invention is MM, examples of the therapeutic antibody for treating include an anti-CS-1 antibody (such as HuLuc63), an anti-CD40 antibody (such as SGN-40 and HCD122), an anti-VEGF antibody (such as bevacizumab), an anti-IL-6 antibody (such as CNTO328) and the like. Examples of the antibody which enhances immune activity include anti-PD-1 antibody, anti-CTLA4 antibody, anti-CCR4 antibody and the like.

The pharmaceutical composition of the present invention can be used for diseases relating to GM2. For example, the pharmaceutical composition can be used for a tumor which expresses GM2 regardless of the kinds of the cancer. As an example of a tumor, MM is included.

The effect of the pharmaceutical composition of the present invention may be examined by measuring an in vivo antitumor activity using animal models.

Examples of the animal models include xenograft models obtained by transplanting a culture cell line derived from a human cancer tissue into mice. The xenograft models can be obtained by transplanting a human cancer cell line into various regions of immunodeficient mice, such as SCID mice, for example, subcutaneously, intracutaneously, intraperitoneally, or intravenously.

The effect of the pharmaceutical composition of the present invention can be evaluated by comparing an effect of administration of the antibody alone or an effect of administration of the agent alone with an effect of the pharmaceutical composition of the present invention by using the above animal models.

The pharmaceutical composition of the present invention can be administered alone, but it is generally preferred to provide it in the form of a pharmaceutical preparation produced by mixing it with one or more pharmaceutically acceptable carriers in accordance with any method well known in the technical field of pharmaceutics.

It is preferable to select a route of administration which is most effective in treatment. Examples include oral administration and parenteral administration, such as intraoral, tracheobronchial, intrarectal, subcutaneous, intramuscular and intravenous. In a protein preparation, intravenous administration is preferred.

Examples of the preparation suitable for the oral administration are spray, capsule, tablet, granule, syrup, emulsion, suppository, injection, ointment, tape and the like.

The pharmaceutical preparations suitable for oral administration include emulsions, syrups, capsules, tablets, powders and granules.

Liquid preparation such as emulsion and syrup can be produced using water, saccharides such as sucrose, sorbitol and fructose, glycols such as polyethylene glycol and propylene glycol, oils such as sesame oil, olive oil and soybean oil, antiseptics such as p-hydroxybenzoate, flavors such as strawberry flavor and peppermint flavor and the like, as additives.

Capsule, tablet, diluted powder, granule, and the like can be produced using excipients such as lactose, glucose, sucrose and mannitol, disintegrating agents such as starch and sodium alginate, lubricants such as magnesium stearate and talc, binders such as polyvinyl alcohol, hydroxypropyl cellulose and gelatin, surfactants such as fatty acid ester, plasticizers such as glycerol, as additives.

Examples of the preparation suitable for parenteral administration are injection, suppository, air spray and the like.

Injections can be prepared using carriers comprising a salt solution, a glucose solution, or a mixture thereof, etc.

Suppository is prepared using a carrier such as cacao butter, hydrogenated fat or carboxylic acid,

Air spray is prepared using the pharmaceutical composition as such or using, for example, a carder which does not stimulate the mouth and the airway mucous membrane of a person to be administered, and which disperses the pharmaceutical composition into fine particles and makes the absorption easy.

Specific examples of the carrier are lactose and glycerol. Depending upon the property of the pharmaceutical composition and the carrier used, it is possible to prepare aerosol, dry powder, and the like. In addition, even in the parenteral preparation, components exemplified as additives in the oral preparation may be added.

A dose or an administration schedule varies depending on a desired therapeutic effect, an administration method, a therapeutic period, an age, a body weight and the like. The dose of the antibody in one administration is usually from 0.1 to 20 mg/kg for an adult. The agent used in combination with the antibody is administered at a dose equal to or Lower than the dose when the agent is used alone in clinic.

According to the present invention, a pharmaceutical composition comprising a combination of a recombinant antibody which specifically binds to GM2 or an antibody fragment thereof and at least one agent is provided.

### EXAMPLE 1

Antitumor Effect Provided by Administrating an Anti-GM2 Antibody and Melphalan in Combination

KMS-11 cells (human multiple myeloma cell; JCRB1179) were suspended in Dulbecco's phosphate buffered saline without calcium chloride and magnesium chloride (PBS, Invitrogen) at a density of 1×10⁸ cells/mL, and 100 μL of the suspension was transplanted into the ventral skin of SCID mouse (Nippon Crea, male). Ten days after the cell transplantation, a diameter of a tumor was measured with calipers, and a tumor volume was calculated using the following formula.

(Formula) Tumor volume=short diameter×short diameter long diameter×0.5

Individuals having the tumor volume within the range of 140 to 184 mm³ were selected, and grouped such that the average of tumor volume was to be almost the same. Each of following administration groups A to D was administered to the mice. Incidentally, the grouped day was defined as Day 0.

A. Negative control group: Administration of Saline
B. Group administering anti-GM2 antibody KM8969 (U.S. Pat. No. 6,872,392) solely: 1 mg/kg was administered on Day 0, Day 3, Day 7, Day 10, Day 14 and Day 17.
C. Group administering Melphalan (hereinafter referred to as L-PAM; Sigma) solely: 3 mg/kg was administered on Day 0 and Day 7.
D. Group administering anti-GM2 antibody KM8969 and L-PAM in combination: the respective agent was administered on the same schedule and at the same dose as each of the agent-alone group.

The experiment was conducted with groups each consisting of five mice. Each of the agents was diluted with a physiological saline (Otsuka Pharmaceutical), and the diluent was administered from the tail vein. With time, the tumor volume was measured. The antitumor effect was evaluated by comparing average values of a tumor volume change (V/V0) when the tumor volume on Day 0 in each group was defined as V0.

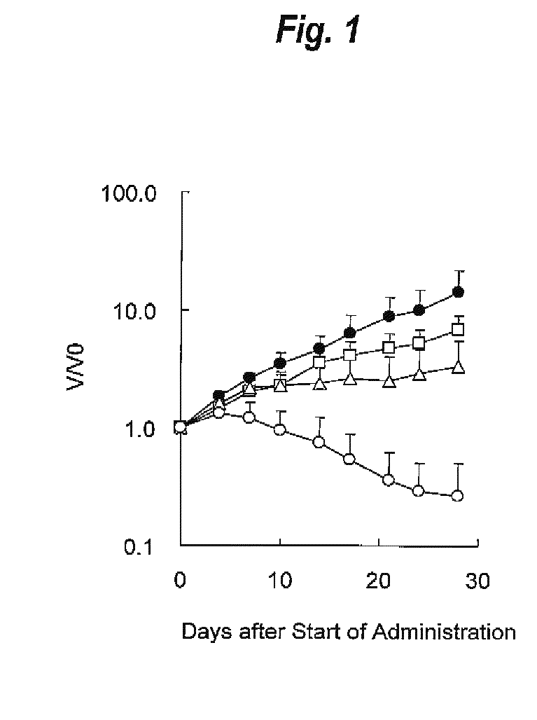

The change over time in the average values of V/V0 in each group is shown in FIG. 1. As shown in FIG. 1, the administration of KM8969 and L-PAM in combination exhibited higher effect for suppressing growth than the administration of L-PAM alone or the antibody alone.

A value (T/C) obtained by dividing V/V0 of each group by V/V0 of the negative control group is shown in Table 1. In comparison with a theoretical value of T/C when simply adding the pharmaceutical effects of both KM8969 and L-PAM, namely, a value obtained by multiplying T/Cs of the groups of administering the respective agents alone, actual T/C of the combined administration group (C in the table) exhibited lower values than the theoretical values on Day 7, Day 10, Day 14, Day 21, Day 24 and Day 28.

TABLE 1

| Group | Day 0 | Day 4 | Day 7 | Day 10 | Day 14 |
|---|---|---|---|---|---|
| KM-8969 | 1.0000 | 0.7949 | 0.7730 | 0.6436 | 0.7878 |
| L-PAM | 1.0000 | 0.8624 | 0.8354 | 0.6430 | 0.5255 |
| Theoretical Value | 1.0000 | 0.6855 | 0.6458 | 0.4138 | 0.4140 |
| Measured Value | 1.0000 | 0.7102 | 0.4602 | 0.2705 | 0.1639 |

| Group | Day 17 | Day 21 | Day 24 | Day 28 |
|---|---|---|---|---|
| KM-8969 | 0.6490 | 0.5437 | 0.5170 | 0.4717 |
| L-PAM | 0.4159 | 0.2881 | 0.2872 | 0.2348 |
| Theoretical Value | 0.2699 | 0.1566 | 0.1485 | 0.1108 |
| Measured Value | 0.0842 | 0.0412 | 0.0286 | 0.0184 |

From the foregoing, it has been clarified that the administration of anti-GM2 antibody and L-PAM in combination has higher antitumor effect than the administration of anti-GM2 antibody alone or L-PAM alone.

### EXAMPLE 2

Antitumor Effect Provided by Administrating an Anti-GM2 Antibody and Bortezomib or Lenalidomide in Combination

The cells (hereinafter referred to as OPM-2/GFP cells) in which GFP gene was introduced into human multiple myeloma cell line OPM-2 cells (obtained from Anticancer) were suspended in PBS at a density of 5×10⁷ cells/mL, and 200 μL of the suspension was transplanted into the tail vein of SCID mice (Nippon Crea, male). The day on which the tumor was transplanted was defined as Day 0. On Day 19 after the cell grafting, M++ protein in blood (human Igλ chain secreted by OPM-2 cell) was measured and mice were grouped into 6 groups such that the average M-protein concentration of each group to be almost the same. Each group was defined as follows.

A. Negative control group: Administration of saline
B. Group administering KM8969 solely: 10 mg/kg was administered on Day 20, Day 23, Day 27, Day 30, Day 34, Day 37, Day 41 and Day 44.
C. Group administering Lenalidomide solely: 1 mg/kg was administered from Day 20 to Day 33.
D. Group administering Bortezomib solely; 0.5 mg/kg was administered on Day 20, Day 23, Day 27, Day 30, Day 34 and Day 37.
E. Group administering KM8969 and Lenalidomide in combination: the respective agent was administrated on the same schedule and at the same dose as each of the agent-alone group.
F. Group administering KM8969 and Bortezomib in combination: the respective agent was administrated on the same schedule and at the same dose as each of the agent-alone group.

The experiment was conducted with groups each consisting of five mice. Each of the agents was diluted with a physiological saline (Otsuka Pharmaceutical), and the diluent was administered from the tail vein. Lenalidomide was intraperitoneally administered per mouse after suspending in 0.5% MC400. On Day 43, the blood of respective mice was collected and then the average M-protein in blood was measured. On Day 61, the whole-body fluorescence was measured so as to check the existence of OPM-2/GFP cell.

On Day 61, the whole-body fluorescence was measured. From the results, the administration of KM8969 and Lenalidomide in combination exhibited higher antitumor effect than the administration of KM8969 alone or Lenalidomide alone. Furthermore, in a similar way, the administration of KM8969 and Bortezomib exhibited higher effect for antitumor than the administration of KM8969 alone or Bortezomib alone.

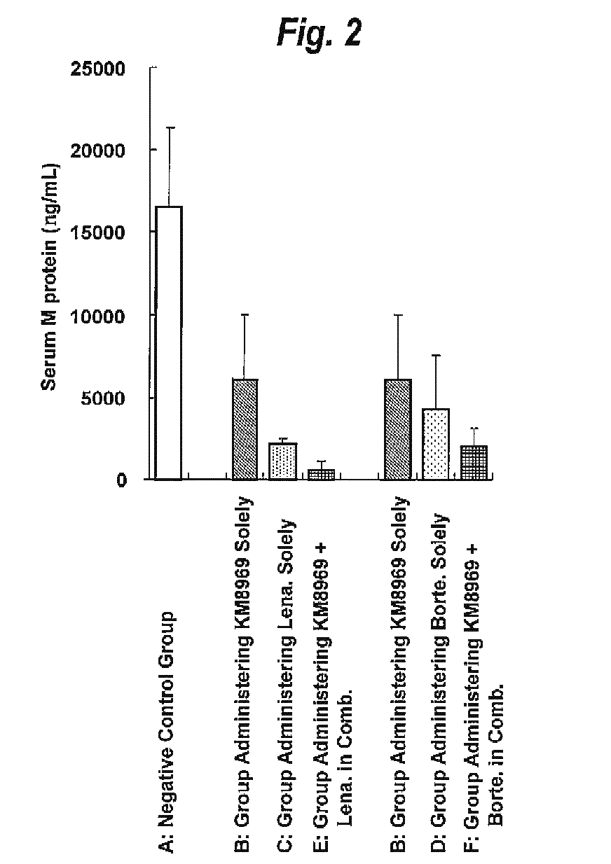

The concentration of M-protein in blood on Day 43 is shown in FIG. 2. The administration in combination strongly inhibited the increase in M-protein in comparison with that of the administration alone. From the foregoing, the administration KM8969 and Lenalidomide in combination and the administration KM8969 and Bortezomib exhibited higher antitumor effect than the administration of each of them alone.

While the present invention has been described in detail and with reference to specific embodiments thereof, it will be apparent to one skill in the art that various changes and modifications can be made therein without departing from the spirit and scope thereof.

This application is based on Japanese application No. 2008-208384, filed on Aug. 13, 2008, and U.S. provisional application No. 61/089,222, filed on Aug. 15, 2008, the entire contents of which are incorporated hereinto by reference. All references cited herein are incorporated in their entirety.

1. A pharmaceutical composition comprising a combination of a recombinant antibody which specifically binds to ganglioside GM2 (GM2) or an antibody fragment thereof and at least one agent.

2. The pharmaceutical composition according to claim 1, which is an antitumor agent.

3. The pharmaceutical composition according to claim 1, wherein the recombinant antibody which specifically binds to GM2 or the antibody fragment thereof is an antibody having at least one activity selected from the group consisting of high ADCC activity and high CDC activity.

4. The pharmaceutical composition according to claim 1, wherein the agent is an agent having a low-molecular weight or an agent having a high-molecular weight.

5. The pharmaceutical composition according to claim 4, wherein the agent having a low-molecular weight is selected from the group consisting of a DNA synthesis inhibitor, a metabolic antagonist, an immuno-modulating agent, a proteasome inhibitor, a steroid agent, an HDAC inhibitor and an Hsp90 inhibitor.

6. The pharmaceutical composition according to claim 2, the antitumor agent is a medicament for multiple myeloma.

7. A method for treating a tumor which expresses GM2, comprising administering a recombinant antibody which specifically binds to ganglioside GM2 (GM2) or an antibody fragment thereof and at least one agent in combination.

8. The method according to claim 7, wherein the recombinant antibody which specifically binds to GM2 or the antibody fragment thereof and at least one agent are administered either simultaneously or successively.

9. The method according to claim 7, wherein the recombinant antibody which specifically binds to GM2 or the antibody fragment thereof is an antibody having at least one activity selected from the group consisting of high ADCC activity and high CDC activity.

10. The method according to claim 7, wherein the agent is an agent having a low-molecular weight or an agent having a high-molecular weight.

11. The method according to claim 10, wherein the agent having a low-molecular weight is selected from the group consisting of a DNA synthesis inhibitor, a mitotic inhibitor, a metabolic antagonist, an immuno-modulating agent, a proteasome inhibitor, a steroid agent, an HDAC inhibitor and an Hsp90 inhibitor.

12. The pharmaceutical composition according to claim 7, the tumor is multiple 15 myeloma.


SEQUENCE LISTING (22 sequences)

SEQ ID NO: 1 - protein, 5 aa, Mus musculus
DYNMD

SEQ ID NO: 2 - protein, 17 aa, Mus musculus
YIYPNNGGTGYNQKFKS

SEQ ID NO: 3 - protein, 11 aa, Mus musculus
TGHYYGYMFAY

SEQ ID NO: 4 - protein, 10 aa, Mus musculus
SASSSVSYMH

SEQ ID NO: 5 - protein, 7 aa, Mus musculus
STSNLAS

SEQ ID NO: 6 - protein, 9 aa, Mus musculus
QQRSSYPYT

SEQ ID NO: 7 - protein, 120 aa, Mus musculus
EVQLQQSGPELVKPGASVKISCKASGYTFTDYNMDWVKQSHGKSLEWIGYIYPNNGGTGYNQKFKSKATLTVDKSSSTAYMELHSLTSEDSAVYYCATYGHYYGYMFAYWGQGTLVTVSA

SEQ ID NO: 8 - protein, 107 aa, Mus musculus
QIVLTQSPAIMSASPGEKVTITCSASSSVSYMHWFQQKPGTSPKLWIYSTSNLASGVPARFSGSGSGTSYSLTISRMEAEDAATYYCQQRSSYPYTFGGGTKLEIKR

SEQ ID NO: 9 - protein, 125 aa, Mus musculus
EVQLVQSGAEVKKPGASVKVSCKASGYTFTDYNMDWVRQAPGQGLEWMGYIYPNNGGTGYNQKFKSKVTITVDTSTSTAYMELHSLRSEDTAVYYCATYGHYYGYMFAYWGQGTLVTVSSASTKG

SEQ ID NO: 10 - protein, 125 aa, Mus musculus
EVQLVQSGAEVKKPGASVKVSCKASGYTFTDYNMDWVRQAPGQGLEWMGYIYPNNGGTGYNQKFKSRVTITADTSTSTAYMELSSLRSEDTAVYYCARYGHYYGYMFAYWGQGTLVTVSSASTKG

SEQ ID NO: 11 - protein, 108 aa, Artificial Sequence
  Synthetic peptide
DIQLTQSPSSLSASVGDRVTITCSASSSVSYMHWYQQKPGKAPKLLIYSTSNLASGVPSRFSGSGSGTDFTFTISSLQPEDIATYYCQQRSSYPYTFGGGTKVEIKRT

SEQ ID NO: 12 - protein, 108 aa, Artificial Sequence
  Synthetic peptide
DIQMTQSPSSLSASVGDRVTITCSASSSVSYMHWYQQKPGKAPKLLIYSTSNLASGVPSRFSGSGSGTDFTLTISSLQPEDFATYYCQQRSSYPYTFGQGTKVEIKRT

SEQ ID NO: 13 - protein, 125 aa, Artificial Sequence
  Synthetic peptide
EVQLVQSGAEVKKPGASVKVSCKASGYTFTDYNMDWVKQSPGQGLEWMGYIYPNNGGTGYNQKFKSKVTITVDTSTSTAYMELHSLRSEDTAVYYCATYGHYYGYMFAYWGQGTLVTVSSASTKG

SEQ ID NO: 14 - protein, 125 aa, Artificial Sequence
  Synthetic peptide
EVQLVQSGAEVKKPGASVKVSCKASGYTFTDYNMDWVKQSPGKSLEWMGYIYPNNGGTGYNQKFKSKVTITVDTSTSTAYMELHSLRSEDTAVYYCATYGHYYGYMFAYWGQGTLVTVSSASTKG

SEQ ID NO: 15 - protein, 125 aa, Artificial Sequence
  Synthetic peptide
EVQLVQSGAEVKKPGASVKVSCKASGYTFTDYNMDWVRQAPGQGLEWMGYIYPNNGGTGYNQKFKSKATLTVDTSTSTAYMELHSLRSEDTAVYYCATYGHYYGYMFAYWGQGTLVTVSSASTKG

SEQ ID NO: 16 - protein, 125 aa, Artificial Sequence
  Synthetic peptide
EVQLVQSGAEVKKPGASVKVSCKASGYTFTDYNMDWVKQSPGKSLEWMGYIYPNNGGTGYNQKFKSKATLTVDTSTSTAYMELHSLRSEDTAVYYCATYGHYYGYMFAYWGQGTLVTVSSASTKG

SEQ ID NO: 17 - protein, 125 aa, Artificial Sequence
  Synthetic peptide
EVQLVQSGAEVKKPGASVKVSCKASGYTFTDYNMDWVKQSPGQGLEWMGYIYPNNGGTGYNQKFKSKATLTVDTSTSTAYMELHSLRSEDTAVYYCATYGHYYGYMFAYWGQGTLVTVSSASTKG

SEQ ID NO: 18 - protein, 108 aa, Artificial Sequence
  Synthetic peptide
DIQLTQSPSSLSASPGDRVTITCSASSSVSYMHWFQQKPGKAPKLWIYSTSNLASGVPARFSGSGSGTSYSLTISRLQPEDIATYYCQQRSSYPYTFGGGTKVEIKRT

SEQ ID NO: 19 - protein, 108 aa, Artificial Sequence
  Synthetic peptide
DIQLTQSPSSLSASPGDRVTITCSASSSVSYMHWFQQKPGKAPKLWIYSTSNLASGVPSRFSGSGSGTSYSLTISRLQPEDIATYYCQQRSSYPYTFGGGTKVEIKRT

SEQ ID NO: 20 - protein, 108 aa, Artificial Sequence
  Synthetic peptide
DIQLTQSPSSLSASPGDRVTITCSASSSVSYMHWFQQKPGKAPKLWIYSTSNLASGVPSRFSGSGSGTSYSFTISSLQPEDIATYYCQQRSSYPYTFGGGTKVEIKRT

SEQ ID NO: 21 - protein, 108 aa, Artificial Sequence
  Synthetic peptide
DIQLTQSPSSLSASPGDRVTITCSASSSVSYMHWYQQKPGKAPKLWIYSTSNLASGVPSRFSGSGSGTSYSLTISRLQPEDIATYYCQQRSSYPYTFGGGTKVEIKRT

SEQ ID NO: 22 - protein, 108 aa, Artificial Sequence
  Synthetic peptide
DIQLTQSPSSMSASPGDRVTITCSASSSVSYMHWFQQKPGKSPKLWIYSTSNLASGVPSRFSGSGSGTSYSLTISSMQPEDFATYYCQQRSSYPYTFGQGTKLEIKRT